(19) United States
(12) Patent Application Publication
(10) Pub. No.: US 2024/0123160 A1
(43) Pub. Date: Apr. 18, 2024

(54) ELECTRO-MECHANICAL INJECTION SITE PREPARATION SYSTEM

(71) Applicant: Jacob Hyer, Apex, NC (US)

(72) Inventor: Jacob Hyer, Apex, NC (US)

(21) Appl. No.: 18/484,802

(22) Filed: Oct. 11, 2023

Related U.S. Application Data
(60) Provisional application No. 63/415,323, filed on Oct. 12, 2022.

(51) Int. Cl. A61M 5/42; A61M 99/00

(52) U.S. Cl. CPC A61M 5/422; A61M 5/425; A61M 99/00; A61M 2205/054; A61M 2205/33; A61M 2209/088; A61M 2210/083

(57) ABSTRACT

The present disclosure relates to a device to provide a discomfort-reducing sensations to an area in preparation for injection or other needle puncture. The system includes a vibration source, as well as an electrical stimulation source. These two operate in combination to cause a numbing sensation to the patient, at which point a needle can puncture the skin without causing pain, discomfort, flinching, and mental distress.

### TECHNICAL FIELD

The present invention relates to preparing sites prior to and during the use of venipuncture, percutaneous injections, or other injections of medicaments.

### BACKGROUND

Venipuncture and injection procedures are common medical practices employed for various diagnostic and therapeutic purposes, such as blood collection, medication administration, and intravenous therapies. This disclosure pertains to innovative techniques aimed at minimizing the pain and discomfort experienced by patients during these procedures.

When preparing a site for venipuncture or percutaneous injection, various methods have been deployed in an attempt to minimize the pain to the patient. Some of these methods include using a topical anesthetic in an attempt to numb the injection site. Others will distract the patient asking the patient questions immediately prior to insertion of the needle or catheter. Unfortunately, these attempts take additional time, are technique sensitive, and are often less effective. Pain from the venipuncture or percutaneous injection may cause the patient to flinch. If the patient flinches: 1) the needle could cause additional pain or harm to the patient, 2) the healthcare provider could potentially suffer harm as the person administering the shot, or 3) the venipuncture or percutaneous injection could be unsuccessful. Fear and anxiety of upcoming appointments due to a fear of needles can lead to avoidance of medical appointments, which is well known to lead to worsening health outcomes for the patient. As such, the present application seeks to improve upon previous techniques and further mitigate flinching and potential harm by providing a system that better prepares sites for injections.

Numerous methods and devices have been developed in the past to alleviate the discomfort associated with venipuncture and percutaneous injection. These methods include the application of topical anesthetics, warming or cooling the skin surface, and utilizing distraction techniques. Additionally, various devices have been designed to enhance the visibility of veins, making it easier to locate and access them. While these existing techniques and devices have been somewhat effective in reducing patient discomfort, they often have limitations, including inconsistent results, increased procedural time, and the potential for side effects associated with anesthetic agents.

Therefore, there exists a need for an improved method and device for preparing a site for venipuncture or percutaneous injection that offers a high degree of effectiveness in reducing patient discomfort, is applicable across diverse patient populations, minimizes the risk of complications, and is user-friendly for healthcare providers.

### SUMMARY

The present disclosure addresses the shortcomings of the existing techniques and devices by providing an innovative approach to prepare a site for needle puncture. This disclosure introduces novel methods and devices designed to minimize patient pain and anxiety, improve procedural efficiency, and enhance patient outcomes. By incorporating cutting-edge technologies and ergonomic design principles, the invention offers a versatile solution that can be easily adopted by healthcare providers in a wide range of medical settings.

The present application relates to an electro-mechanical system for preparing injection sites, the system comprises a framework, wherein the framework comprises a framework first side, a framework second side, and a framework adjustable side; a vibratory system comprising at least a vibration motor; a power supply connected to the framework; an electrical stimulation system, wherein a portion of the electrical stimulation includes electrical stimulating areas disposed on each of the framework first side and framework second side; a first set of controls disposed in a control unit for operating the vibratory system and the electrical stimulation system; and a wire connecting the power supply and the framework.

The electro-mechanical system further comprises an adjustable strap to hold the framework in place on an arm, wrist, or other similar area, wherein the adjustable strap has two ends; one end connected to the framework first side and the other end connected to the framework second side. In addition, the system may further comprise a tourniquet strap which may also hold the power supply and the control unit a short distance from the framework. A cord retention post can be added to the controller unit to secure the wire connecting the power supply to the framework. In one embodiment, the adjustable strap may operate as a tourniquet as well, and in other embodiments the adjustable strap and tourniquet are separate.

Of course, the present invention is not limited to the above features and advantages. Those of ordinary skill in the art will recognize additional features and advantages upon reading the following detailed description, and upon viewing the accompanying drawings.

### BRIEF DESCRIPTION OF THE DRAWINGS

FIG. 1 depicts a perspective view of a framework of an electro-mechanical system used to prepare a venipuncture or an injection site on an arm.

FIG. 2 depicts a top view of a framework of an electro-mechanical system used to prepare a venipuncture or an injection site on a hand.

FIG. 3 depicts a bottom view of a framework of an electro-mechanical system along with an adjustable strap.

FIG. 4 depicts an electro-mechanical system used to prepare injection sites in use on a patient's arm.

### DETAILED DESCRIPTION

Embodiments of the present invention are directed to an electro-mechanical system used to prepare injection sites and methods of using the same.

As noted above, one of the objectives is to reduce pain and sensation of an injection site while administering an injection. This injection site could be anywhere on the human body including in the wrist, forearm, shoulder, back of the hand, or foot. There is a Gate Control Theory of Pain that essentially suggests non painful stimulus can override painful stimulus. In other words, neuronal input from non-noxious stimulation closes the gate on the noxious neuronal input on higher levels of pain.

It has been documented that transcutaneous electrical nerve stimulations (‘TENS’) uses electric current produced by a device to stimulate the nerves. This can be used for various purposes including eliminating pain. TENS and ultra-low frequency TENS can be used to treat acute or chronic pain. In some ways, TENS use can stimulate competing sensory neurons at the pain reception gate as well as stimulate the opiate response. Other similar electromagnetic systems may also be effective to stimulate nerves and are within the scope of this disclosure.

Vibration and vibrator mechanisms can also have an overwhelming impact on the sensory system, which similar to TENS, can overcome or stimulate competing sensory neurons, as well as stimulate the opiate response. The present embodiments seek to combine the benefits of vibration and TENS into a single electromechanical system. The combined operation of the two systems leads to even better results than one system alone, and appears to provide synergistic advantages beyond what was expected indicating unexpected results from this combination.

The framework may be formed of any material to support the vibration devices (typically a vibration motor) and electrical stimulation system contacts (typically, though not necessarily, electrodes), as well as wiring connecting both to a power supply and controller. The framework is typically formed in a three-sided configuration such as an approximate “U” shape with the first side and second side extending as two arms away from the adjustable side which acts as a base to connect the two sides. In other embodiments, the framework may define a closed space such as a rectangle, or other open space, without straying from the scope of this disclosure. The adjustable side and first and second sides define a stimulation area between them in which a skin can be stimulated so as to be painlessly pierced by a needle.

The vibratory system or vibration system may be any vibrator operable to cause vibrations rapid enough to stimulate the nerves in adjacent skin. Typically this vibrator utilizes a vibrating motor or other equivalent device. Other non-motorized vibration systems are also contemplated herein. The vibration system is sized and configured to fit on a surface of the framework or within the framework, such as on the framework first side, second side, or adjustable side, or combinations thereof. In certain embodiments, the vibration system may be within the framework or on one end of the framework. In a further embodiment, the framework may define protrusions, or nubs on a skin contacting surface to increase force transfer to the skin.

The electrical stimulation system is, in most embodiments, a TENS system as described herein. As also noted, other equivalent or similar electrical stimulation systems may be used which are able to electrically stimulate nerves in adjacent skin. The electrical stimulation system may have contacts, such as electrodes, on the surface of the framework, typically on a skin-contacting side. Electrodes may be positioned on or in on the framework first side, second side, or adjustable side, or combinations thereof. In other embodiments, the contacts or electrodes may be within the framework and able to stimulate adjacent nerves in skin through the material of the framework, or a thin layer thereof.

The power source and controller may be any structures capable of delivering adequate power to the vibration system and electrical stimulation system. The power source may be a wall plug connection, other plug connection, or battery power source. The controller may be any device having a user input or interface to allow control of operation, including optionally control of operational parameters such as speed and intensity of one or both of the vibration system and electrical stimulation system.

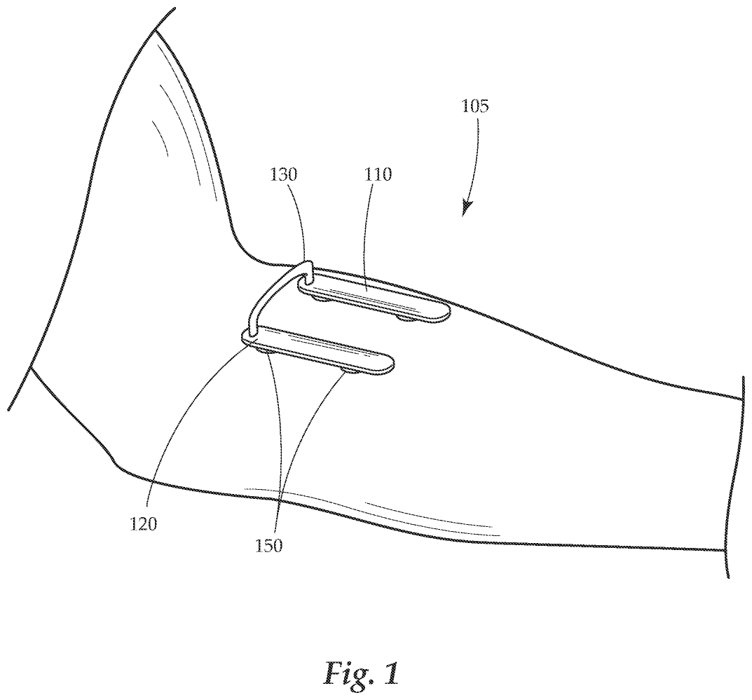

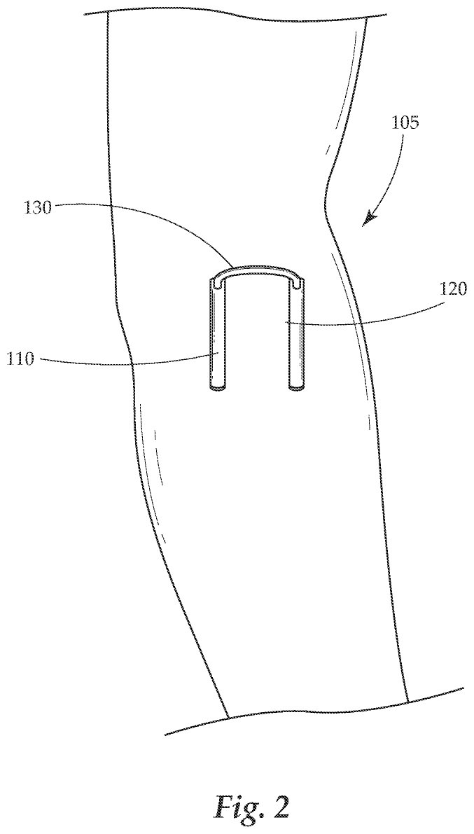

FIGS. 1-4 illustrate various views of an electro-mechanical system that can be used for venipuncture or injection site preparation and administration. The system includes a framework 105 that forms a framework first side 110, a framework second side 120, a framework adjustable side 130, one or more vibration motors 145, and one or more electrical stimulating electrode 150, shown here as electrodes of a TENS device. The system further has a controller unit 170, wherein the controller unit 170 comprises a power supply 185, and a wire 190. The system can also include an adjustable strap 160, a tourniquet strap 180, and a cord retention post 195.

The controller unit 170 can include a set of operating controls 172 to allow a user to control operation of the device. Electrical stimulating electrode 150 can be positioned at or near the ends of the framework first side 110 and the framework second side 120. The adjustable side 130 is adjustable in length to accommodate differently sized body parts and/or patients. Adjustable length structures may be any structures capable of expanding or contracting relative to each other. For example, a telescoping structure, slidable arms which can overlap at varying extents, ratcheting structures with a release tab, elastic or stretchable materials, combinations thereof, and the like. Other non-adjustable embodiments are also contemplated herein which may be more simple to manufacture, assemble, and/or use. In such an embodiment, the “adjustable side” may be termed as “framework base” as the other sides 110, 120 attach to the base and extend therefrom.

The vibrator, shown here as a vibration motor 145 can be attached to the electrical stimulating electrodes 150, directly adjacent and in contact therewith, or can be be attached individually on the framework 105, among other options. The framework adjustable side 130 has a length that is adjustable to fit on arms, legs, wrists, or other areas of the body of varying circumference. The framework adjustable side 130 has a locking mechanism 135 to hold it in a fixed position during use. In the embodiment shown, the adjustable strap 160 comprises a first end 162 and second end 164 wherein first end 162 is removably or permanently attached to framework first side 110 and second end 164 is removably or permanently attached to framework second side 120. Other forms of connection of the framework to the body of a patient may also be used, for example by adhesive or tape, physically holding the device in place, and the like.

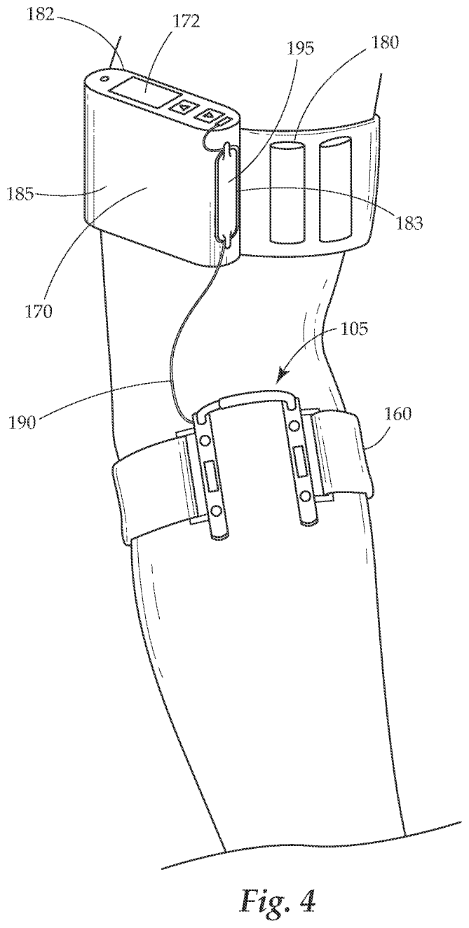

A tourniquet strap 180 comprises a first end 182 and a second end 183 wherein either the first end or the second end may have a quick release 184. The tourniquet strap 180 may facilitate securing an inserted cannula or needle to its anatomical location on the patient, for location of a proper injection or piercing location, and the like. The tourniquet strap 180 is capable of temporarily securing the controller unit 170 and power supply 185 to the appendage where the injection site is located. For example, if the injection site is in the arm, the tourniquet strap 180 can be secured to the upper portion of the arm while the adjustable strap 160 along with the framework 105 is secured to the lower portion of the arm (see FIG. 4). Of course, in other embodiments, the controller unit 170 and/or power supply 185 may be located elsewhere. For example, they may be integrated into the framework 105, remote from the framework 105 and connected wirelessly or via a wire, and the like.

The controller unit 170 can cause the vibration motor 145, and thus the framework first side 110 and the framework second side 120, to oscillate or vibrate. The controls of the controller unit 170 can be used to modify or change the vibratory pattern, intensity, and frequency. The first set of controls can also be used to control the intensity and frequency of the electrical stimulator electrodes 150, which, when touching an injection site area, function as a TENS system. Thus, system includes both vibratory and TENS functionality that can be operated individually, sequentially (one after another, repeating), or simultaneously in the preparation of the injection site. The controller interface may allow a user to select between simultaneous operation of the vibratory and TENS units, sequential operation, and individual operation. In further embodiments, the controller may have one or more pre-programmed operational conditions such as a sequential activation of the vibratory system for a certain time, activation of the TENS unit for a certain time, and repeating in this order. The sequential operation may have some or no overlap in operation of one and the other. It has been unexpectedly found that the use of both vibration and electrical stimulation provides improved effectiveness compared to one or the other, indicating a synergistic effect that cannot be achieved by only one system. Depending on the sensitivity of the area, it may be advantageous to use a simultaneous activation of both systems, while in other areas, the sequential use may be used, which still achieves some of the noted synergistic effects while reducing power usage.

For example, in one method of using the system, the method can include first vibrating the injection site with a first intensity and a first frequency, after a period of applying the first vibrations, the administrator can trigger the set of controls to begin operating the electrical stimulators 150, which function as a TENS system. At this point the administrator, or an assistant to the administrator can insert or inject needle at the injection site and deploy an intravenous cannula, perform phlebotomy, or deliver the desired medicament. The administrator can then turn off the TENS system and then turn off the vibratory system. Alternatively, they can be simultaneously turned off.

A variation to the above method includes modifying the first intensity and frequency of the vibratory system of the present disclosure to a second intensity and frequency during the injection or shortly thereafter. The change in vibration can aid in distributing the medicament into the injection site areas, which may speed up the effects of the delivered medicament. In some theories, the more the nerves are stimulated the faster the medicament such as a local anesthetic will take effect.

In yet another variation, the TENS system could be operated first using a first intensity and first frequency, then the vibration system could be turned on while preparing the injection site. A second intensity and frequency (as well as pattern) could be utilized during the injection. The administrator could revert back to the first settings for the preparing of the site or switch to a third set of settings for post-injection treatment. Likewise, the vibration system could include a first, second, third or more sets of settings, each being used at an appropriate time during the pre-injecting, injecting, and post-injecting phases or different patient tolerances. It should be noted that some patients can tolerate higher or lower intensities better, so the frequencies of either the mechanical vibration or electrical stimulation can alter according to the individual patient's need.

In another embodiment of use, a quantity of sterile saline solution may be applied, coated, or otherwise disposed on skin on and around the injection site. Preferably the saline is applied near the area of the skin stimulated by the TENS system and its electrodes. This enhances the conductivity of the electrical signal across the surface of the skin, making the TENS system more effective. Other similar components such as liquids, gels and even solids that can increase conductivity may also be used to the same or similar effect.

These settings can also be altered depending on where the injection site is on the patient. For example, back of the hand settings might require much lower intensity TENS settings as the sensitivity is higher in contrast to an area on the antecubital fossa.

In a particular embodiment, the controller may have one or more pre-programmed patterns of operations. As noted, changes in operational pattern can further aid in making the needle puncture less distressing. The system may also be programmed to provide an indication such as a noise or light to the operator to indicate when is an optimal time to insert the needle. In one embodiment, the pre-programmed pattern of operation may have a preparation phase, injection phase for best injection, and a cooldown phase to help disperse injected medicine and/or relax the patient. In another embodiment, the pre-programmed pattern may have only the preparation phase and injection phase.

It should also be noted the electrical stimulators 150 can be formed of a different material from the rest of the framework 105. For example, they could be formed of a metallic or other electrically conductive material, whereas the framework could be formed of a durable yet either rigid, or resilient, plastic material.

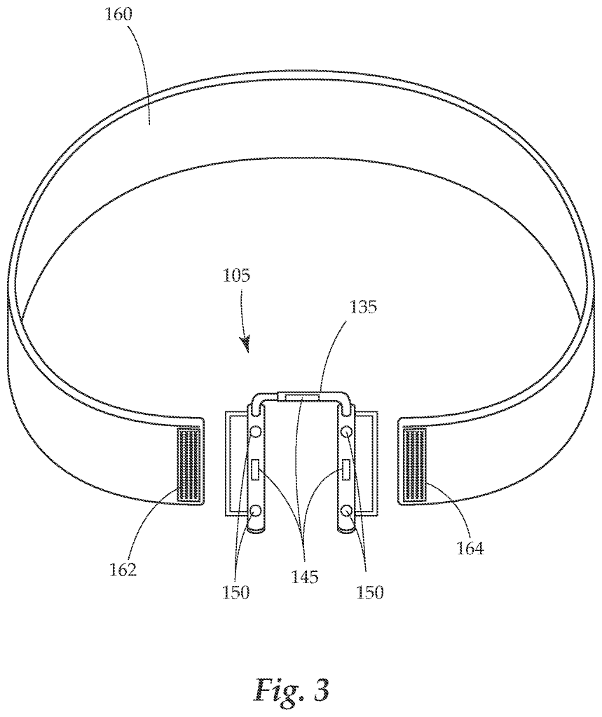

It is desirable that the framework 105 be formed of a material that has sufficient rigidity to translate the vibrations generated from the vibration motor 145, which in most embodiments is disposed in framework 105. In one embodiment, the framework 105 will have one vibrator, shown here as a vibration motor 145 which may convey vibrations to both the framework first side and second side, while in another embodiment the framework may have multiple vibrators, such as a vibration motor 145 on the framework first side, and another vibration motor 145 on the framework second side. In further multiple vibrator embodiments, the system may have vibration motors 145 on framework first side, framework second side, and framework adjustable side, as can be seen in, for example, FIG. 3.

Of course, the present invention is not limited to the above features and advantages. Those of ordinary skill in the art will recognize additional features and advantages upon reading the following detailed description, and upon viewing the accompanying drawings.

Notably, modifications and other embodiments of the disclosed invention(s) will come to mind to one skilled in the art having the benefit of the teachings presented in the foregoing descriptions and the associated drawings. Therefore, it is to be understood that the invention(s) is/are not to be limited to the specific embodiments disclosed and that modifications and other embodiments are intended to be included within the scope of this disclosure. Although specific terms may be employed herein, they are used in a generic and descriptive sense only and not for purposes of limitation.

1. A device for preparing a skin tissue site for needle puncture comprising:
a framework, the framework comprising a framework first side, a framework second side, and a framework adjustable side, the framework first side and framework second side each connected to the framework adjustable side, and the framework first side and framework second side defining a space between each other;
wherein the framework is sized and configured to be attached to a body adjacent to skin;
a vibrator connected to the framework and operable to convey vibrations to at least one of the framework first side and the framework second side;
an electrical stimulation device having a portion connected to the framework and operable to electrically stimulate nerves in tissue.

2. The device for preparing a skin tissue site for needle puncture of claim 1 wherein the vibrator is on the framework first side and comprising a second vibrator on the framework second side.

3. The device for preparing a skin tissue site for needle puncture of claim 1 wherein the vibrator is on the framework adjustable side.

4. The device for preparing a skin tissue site for needle puncture of claim 1 wherein the electrical stimulation device is a transcutaneous electrical nerve stimulation device.

5. The device for preparing a skin tissue site for needle puncture of claim 1 wherein the electrical stimulation device comprises a first electrode on a skin-facing side of the framework first side, and a second electrode on a skin facing side of the framework second side.

6. The device for preparing a skin tissue site for needle puncture of claim 5 wherein the first electrode is on an outer surface of the framework first side, and wherein the second electrode is on an outer surface of the framework second side.

7. The device for preparing a skin tissue site for needle puncture of claim 1 further comprising a power supply operable to provide electrical power to the vibrator and to the electrical stimulation device.

8. The device for preparing a skin tissue site for needle puncture of claim 1 further comprising a controller, the controller in electronic communication with both the vibrator and the electrical stimulation device and operable to control an operation of each.

9. The device for preparing a skin tissue site for needle puncture of claim 7 further comprising a controller, the controller in electronic communication with both the vibrator and the electrical stimulation device and operable to control an operation of each.

10. The device for preparing a skin tissue site for needle puncture of claim 8 wherein the controller is programmed to activate and deactivate the vibrator and electrical stimulation device simultaneously.

11. The device for preparing a skin tissue site for needle puncture of claim 8 wherein the controller is programmed to activate and deactivate the vibrator and electrical stimulation device sequentially.

12. The device for preparing a skin tissue site for needle puncture of claim 1 further comprising a tourniquet.

13. The device for preparing a skin tissue site for needle puncture of claim 12 further comprising a power supply attached to the tourniquet, the power supply operable to provide electrical power to the vibrator and to the electrical stimulation device, and further comprising a controller attached to the tourniquet, the controller in electronic communication with both the vibrator and the electrical stimulation device and operable to control an operation of each.

14. The device for preparing a skin tissue site for needle puncture of claim 1 further comprising an adjustable strap connected to the framework.

15. The device for preparing a skin tissue site for needle puncture of claim 9 further comprising a cord retention post attached to the controller, the cord retention post configured to secure a wire connecting the power supply to the framework.

16. A skin stimulation system comprising:
a framework, the framework comprising a framework first side, a framework second side, and a framework adjustable side, the framework first side and framework second side each connected to the framework adjustable side, and the framework first side and framework second side defining a space between each other;
wherein the framework is sized and configured to be attached to a body adjacent to skin;
a vibrator connected to the framework and operable to convey vibrations to at least one of the framework first side and the framework second side;
an electrical stimulation device having a portion connected to the framework and operable to electrically stimulate nerves in tissue;
wherein the electrical stimulation device is a transcutaneous electrical nerve stimulation device, wherein the electrical stimulation device comprises a first electrode on an outer surface of a skin-facing side of the framework first side, and a second electrode on an outer surface of a skin facing side of the framework second side;
a power supply operable to provide electrical power to the vibrator and to the electrical stimulation device;
a controller, the controller in electronic communication with both the vibrator and the electrical stimulation device and operable to control an operation of each, wherein the controller has a first programming operable to activate and deactivate the vibrator and electrical stimulation device simultaneously, and has a second programming operable to activate and deactivate the vibrator and electrical stimulation device sequentially.

17. The system of claim 16 wherein the vibrator is on the framework first side and comprising a second vibrator on the framework second side.

18. The system of claim 16 wherein the vibrator is on the framework adjustable side.

19. A method of operating the device of claim 1 comprising the steps of:
attaching the framework to a patient at an area of the patient that will receive a needle puncture;
activating the vibrator;
activating the electrical stimulation device;
puncturing a skin of the patient with the needle while at least one of the vibrator and electrical stimulation device is activated.

20. The method of claim 19 further comprising the step of applying a saline solution to skin at or close to the electrical stimulation device of the patient before the step of activating the electrical stimulation device.
